(19) United States
(12) Patent Application Publication
(10) Pub. No.: US 2018/0185411 A1
(43) Pub. Date: Jul. 5, 2018

(54) TOPICAL VAGINAL COMPOSITION

(71) Applicant: CINDY BELANCE, RANDOLPH, MA (US)

(72) Inventor: CINDY BELANCE, RANDOLPH, MA (US)

(21) Appl. No.: 15/396,649

(22) Filed: Dec. 31, 2016

(51) Int. Cl. A61K 33/06; A61K 9/00; A61K 47/12; A61K 31/201; A61K 9/06

(52) U.S. Cl. CPC A61K 33/06; A61K 9/0034; A61K 9/06; A61K 31/201; A61K 47/12

(57) ABSTRACT

A topical vaginal pharmacological preparation for application in contact with vaginal walls which includes potassium aluminum sulfate. In some forms of the present invention the topical vaginal pharmacological preparation of further includes palmitic acid that is added in this product to adjust texture and as a thickening agent and provide lubrication as a release agent. The topical vaginal preparation may further including linoleic acid to adjust texture, reduce viscosity and to reduce sold fat content at room temperature as well as being an anti-inflammatory, anti-oxidative protection and a moisture retention additive.

### TECHNICAL FIELD

The present invention relates to the field of gynecology and particularly to topical applications intended to contract the vagina and thereby enhance sexual satisfaction of sexual partners. More specifically, the present invention relates to topical vaginal preparations Intended to temporarily contract the walls of the vagina.

### BACKGROUND OF THE INVENTION

Surgical procedures for vaginal tightening, referred to as a vaginoplasty are done under general anesthesia and involve removing some of the mucosa, tightening the muscle layer, and reducing the size of the vaginal passageway. Such surgical procedures have reportedly an average cost of between $5500 and $6000.

Vaginoplasty is a procedure for tightening the vagina which has become loosened. Childbirth is usually the cause of vaginal laxity, but women who've never had children or never had a vaginal delivery can also develop vaginal laxity. The complexity of the surgical procedure depends on how much laxity is present and whether there has been any damage to the pelvic floor supports of the bladder, the uterus and the rectum.

It is an object of the present invention to provide a compound capable of inducing sustained contraction of the vaginal wall with a purpose of increasing the contract-friction and thereby resulting a more pleasurable sexual experience for female and male alike.

It is an object of the present invention to provide the female with a compound capable of inducing sustained contraction of the vaginal wall with a purpose of increasing friction with direct penis resulting in a more pleasurable sexual experience for female and male alike.

It is an object of the present invention to provide the female with a pharmacological compound capable of inducing sustained contraction of the vaginal wall with a purpose of increasing the contract-friction with an erected penis, resulting in a more pleasurable sexual experience for female and male alike.

The applicant has identified a topical preparation which when applied into the vagina and placed in contact with the vaginal walls, induces a short lived contraction of the smooth muscle of the vaginal walls causing overall tightening of the vaginal tube/canal. The topical preparation may be in the form of a cream or drops or dissolving vaginal tablets or suppositories.

With proper dosage, contraction of the smooth muscle of the vagina can be controlled in term of time and strength of contraction.

### SUMMARY OF THE INVENTION

The shortcomings of the prior art are overcome and additional advantages are provided through a topical vaginal pharmacological preparation for application in contact with vaginal walls which includes potassium aluminum sulfate.

In some forms of the present invention the topical vaginal pharmacological preparation of further includes palmitic acid that is added in this product to adjust texture and as a thickening agent and provide lubrication as a release agent. The topical vaginal preparation may further including linoleic acid to adjust texture, reduce viscosity and to reduce sold fat content at room temperature. as well as being an anti-inflammatory, anti-oxidative protection and a moisture retention additive.

In some embodiments, the topical vaginal preparation further includes at least one of the acids selected from the group consisting of stearic acid, myristic acid and capric acid. The topical vaginal preparation may be in the form a cream, intravaginal drops, intravaginal dissolving tablets or an elongated cylindrical body.

Additional features and advantages are realized through the techniques of the present invention. Other embodiments and aspects of the invention are described in detail herein and are considered a part of the claimed invention.

The recitation herein of desirable objects which are met by various embodiments of the present invention is not meant to imply or suggest that any or all of these objects are present as essential features, either individually or collectively, in the most general embodiment of the present invention or in any of its more specific embodiments.

### DETAILED DESCRIPTION

A preferred form of the preparation in accordance with the present invention has the following composition:

| | APPROXIMATE |
|---|---|
| INGREDIENT | % by WEIGHT |
| Potassium aluminum sulfate (KAI(SO₄)₂•12H₂O) | 63.00 |
| B Palmitic acid | 3.16 |
| Linoleic Acid | 1.93 |
| Stearic acid | 1.37 |
| Oleic Acid | 1.30 |
| Myristic acid | 0.26 |
| Capric acid | 0.15 |
| Octadecenoic Acid | 0.10 |
| C Lactose monohydrate (C₁₂H₂₂O₁₁•H₂O) | 28.00 |
| D Grassroots extract | 0.73 |

A topical pharmacological preparation in the form of cream, drops, suppositories, or other elongate cylindrical body delivered topically to the vagina in order to contract the smooth muscle of the vaginal wall. The preparation contains a smooth muscle contracting agent capable of constricting the vaginal tube reducing its overall diameter for a more pleasurable sexual experience for the female subject and his partner alike. The formulation achieves high local tissue concentration of the smooth muscle contraction substance in the vicinity of the site of application. As a result of the use of such formulation an increased local smooth muscle contraction activity is expected to occur in the muscularis mucosae.

The following describes with greater particularity the function of the respective ingredients:

Potassium Aluminum Sulfate: It is a strong astringent that tightens the vaginal muscle walls. However, the constant use of alum in the vagina can cause yeast infections and cause irritation if not properly used. In this product, other ingredients are added (such as fatty acids) in order to reduce irritation of skin.

Palmitic Acid: It is a common saturated fatty acid found in plant oil and animal fat. It is a high melting point fatty acid (62.9 degree C.) and is a solid at room temperature. It is commonly used in cosmetic products and added in this product to adjust texture as a thickening agent and provide lubrication as a release agent.

Linoleic Acid: It is a common unsaturated fatty acid found mostly in plant oil. It is a low melting point fatty acid (−7 degree C.) and is a liquid at room temperature. It is added in this product to adjust texture to reduce viscosity and to reduce sold fat content at room temperature. It is widely used in cosmetic products and also in this product because of its multiple beneficial properties for human skin, such as anti-inflammatory, anti-oxidative protection moisture retention activities.

Stearic Acid, Myristic Acid and Capric Acid: These are also common saturated fatty acids found in plant oil and animal fat with melting points lower than that of palmitic acid, but still higher than room temperature (such as 54.4 degree C. for myristic acid and 31.6 degree C. for capric acid). Accurate and fine-tuned formula with different amounts of these fatty acids allow precise control for optimized physical properties, appearance and performance (such as skin penetration and relief from irritation) in the final product.

Oleic Acid, Octadecenoic Acid: These are also common unsaturated fatty acids found in plant oil with melting points lower than that of linoleic acid and with similar beneficial properties. Accurate and fine-tuned formula with different amounts of these fatty acids is necessary for best performance.

Lactose Monohydrate: a common disaccharide found in milk and dairy products. It has lower water solubility than other sugars and is therefore commonly used as a skin-friendly filler or carrier of active ingredients in cosmetic products.

Grassroots Extract: It is a common plant extract commonly used in skin care products, and also in this product for its moisturizing benefits and skin-tightening effects.

In a preferred form of the present invention the composition is provided is provided in the form of the elongated cylinder having rounded axial extremities. Ideally utilization occurs approximately 15 minutes prior to intercourse. The user preferably cleans her hands and vaginal area using a feminine war sure antibacterial soap. Thereafter, substantially one half of the axial extent of the elongated cylinder is wet with warm water and then placed into the vagina. Preferably, the elongated cylinder is manually rotated for 15 to 30 seconds. Is generally is preferable to evaluate the minimum exposure time and gradually increase to a longer time which ordinarily will not exceed 30 seconds.

Upon removal of the elongated cylinder from the vagina the user should again worship hands in vaginal area again preferably with a feminine hygiene soap. Typically, the elongated cylinder will decrease in weight approximately 3 to 5 grams for each application and the effects of the application okay will continue for one to two days depending upon the specific individual involved and sexual lifestyle. Users may a personal lubricant.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the description above contains many specifics, these should not be construed as limiting the scope of the invention, but as merely providing illustrations of some of the presently preferred embodiments of this invention. Thus, the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

1-8. (canceled)

9. A method for vaginal tightening which comprises
providing an elongated object dimensioned and configured for insertion into a vagina having at least the exterior surface which comprises potassium aluminum sulfate;
inserting the elongated object into a vagina; and
manipulating the elongated object with respect to the vagina to increase contact between the exterior surface of the object and the vagina.

10. The method as described in claim 9 wherein the providing step includes providing an elongated object having at least an exterior surface which further includes palmitic acid that is added in this product to adjust texture and as a thickening agent and provide lubrication as a release agent.

11. The method as described in claim 10 wherein the providing step includes providing an elongated object having at least an exterior surface which further includes linoleic acid to adjust texture, reduce viscosity and to reduce sold fat content at room temperature as well as being anti-inflammatory, anti-oxidative protection and a moisture retention additive.

12. The method as described in claim 11 wherein the providing step includes providing an elongated object having at least an exterior surface further including at least one of the acids selected from the group consisting of stearic acid, myristic acid and capric acid.

13. The method as described in claim 9 wherein the providing step includes providing an elongated object having at least an exterior surface that is covered by a cream.

14. the method as described in claim 9 wherein the providing step includes providing the entire physical extent of the elongated object with same composition as the exterior surface thereof.

15. An applicator having a tangible elongated body dimensioned and configured for insertion into a vagina wherein the improvement comprises:
an outer surface thereof is at least partly composed of potassium aluminum sulfate.

16. The applicator as described in claim 15 wherein an outer surface thereof is at least partly composed of including palmitic acid that is added in this product to adjust texture and as a thickening agent and provide lubrication as a release agent.

17. The applicator as described in claim 16 wherein an outer surface thereof is at least partly composed of linoleic acid to adjust texture, reduce viscosity and to reduce sold fat content at room temperature as well as being anti-inflammatory, anti-oxidative protection and a moisture retention additive.

18. The applicator as described in claim 17 wherein an outer surface thereof is at least partly composed of at least one of the acids selected from the group consisting of stearic acid, myristic acid and capric acid.

19. The applicator as described in claim 15 wherein is in the form in the form of an elongated cylindrical body.
